(19) United States
(12) Patent Application Publication
(10) Pub. No.: US 2022/0339323 A1
(43) Pub. Date: Oct. 27, 2022

(54) GRAFT PATCH FOR THE TREATMENT OF MYOPIA AND OPHTHALMIC CONDITIONS

(71) Applicant: CORNEAT VISION LTD, Raanana (IL)

(72) Inventors: Gilad LITVIN, Moshav Sde Varburg (IL); Almog ALEY-RAZ, Moshav Sde Varburg (IL)

(73) Assignee: Corneat Vision Ltd, Raanana (IL)

(21) Appl. No.: 17/640,920

(22) PCT Filed: Sep. 10, 2020
(86) PCT No.: PCT/IL2020/050994

§ 371 Date: Mar. 7, 2022

Related U.S. Application Data
(60) Provisional application No. 62/899,185, filed on Sep. 12, 2019.

(51) Int. Cl. A61L 27/56; A61K 9/00; A61L 27/58; A61L 27/54

(52) U.S. Cl. CPC A61L 27/56; A61K 9/0051; A61L 27/58; A61L 27/54; A61L 2400/12; A61L 2430/16

(57) ABSTRACT

The invention provides a synthetic ophthalmic device comprising a porous polymeric structure; wherein said structure is in the shape of a truncated hemisphere and uses thereof in the treatment of ophthalmic conditions, diseases and syndromes.

### BACKGROUND OF THE INVENTION

The incidence of myopia is increasing worldwide. Myopia is a refractive disorder of the eye. It is estimated that over 285 million people in the world have vision impairment and that 42% of this is due to uncorrected refractive errors. Estimates based on epidemiological studies from 2010 indicate that myopia affected then 1.89 billion people worldwide, and, if the current prevalence rates do not change, projections show that it will affect 2.56 billion people by 2020.

The disorder has significant public healthcare implications worldwide and poses a significant societal and economic burden to healthcare systems globally. The associated increase in secondary and vision-threatening eye diseases will pose major challenges to patients, ophthalmologists, optometrists, opticians and healthcare systems.

Myopia is etiologically heterogeneous, with a low level of myopia of clearly genetic origins that appears without exposure to risk factors Ample evidence supports heritability of most forms of myopia, especially for high grade myopia commonly referred to as high myopia characterized by spherical refractive error of 5-6 D or higher. Recent large genome-wide association studies (GWAS) have identified more than 20 associated loci for myopia. However, the rise in prevalence of high myopia currently has an unusual pattern of development, with increases in prevalence first appearing at approximately age 11. This pattern suggests that the increasing prevalence of high myopia is due to the progression of myopia in children who became myopic at approximately age 6 or 7, and age-specific progression rates typical of East Asia will take these children to the threshold for high myopia in 5-6 years. This high myopia seems to be acquired, whereas high myopia in previous generations tended to be genetic in origin.

Near work was found to be associated with myopia among American children in the Orinda Longitudinal Study of Myopia and in Australian children in the Sydney Myopia Study but was not significantly associated with incident myopia in Singaporean children. Even the results from more recent studies have been equivocal, with some studies showing positive findings, whereas others reported no relationship. A recent meta-analysis found that more time spent on near work activities was associated with a higher risk for myopia [Odds Ratio (OR) 1.14, 95% confidence interval (CI) 1.08-1.20] and that the risk of myopia increased by 2% (OR 1.02, 95% CI 1.01-1.03) for every 1-diopter-h more near work per week. Increasing evidence suggests that the intensity of near work, that is, sustained reading at closer distances (less than 30 cm) with fewer breaks, may be more important than the total hours of near work.

Since myopia begins in childhood and adolescence, progression can only be influenced at this phase of life. Optical and pharmacological treatment options, which show average effect sizes of up to 50% progression reduction, have a comparatively favorable side effect profile. From all methods studied in an attempt to slow the progression of myopia, topical pharmaceutical agents, orthokeratology contact lenses, and soft bifocal contact lenses were found to be the most effective, commercially available modalities. However, none of them are approved by the FDA to slow the progression of myopia.

More invasive methods aimed at the cessation of myopic progression have been tested in animals and comprise mainly of different approaches for fortifying the sclera by external crosslinking techniques. Surgical correction for the complications of myopia comprise of; (i) scleral buckle and Pars Plana Vitrectomy techniques for myopic macular hole, Rhegmatogenous Retinal Detachment, (ii) Avastin and other anti-VEGF intravitreal injection for Choroidal neovascularization, (iii) surgical and pharmacological treatment of associated glaucoma. Surgical treatments aimed at halting the progression of myopia are currently missing from the clinician's arsenal and from the professional literature.

### SUMMARY OF THE INVENTION

The present invention provides scleral fortification using a designated unique design of a patch to strengthen the sclera thus halting the progression of myopia influenced by near work or genetics. This fortification can either come in the form of a simple patch fortifying a specific scleral area; the macular area, to avoid the formation of myopic staphyloma, or as a specially designed form creating a scaffold for fibroblast growth and collagen embedding at an area at risk of thinning thus causing elongation of the eye which in turn causes myopia.

For this purpose, fortification/strengthening of the scleral tissue needs to take place external to the insertion of the ciliary body into the sclera and on the longitudinal axis of the eye thus preventing longitudinal elongation of the globe resulting in myopic progression.

Beside progression of the myopia, myopic patients can suffer from posterior pole/macular scleral thinning. This thinning causes complications such as choroidal neovascularization, macular hole, macular staphyloma, myopic macular detachment and so forth. The described invention might include a portion of scleral reinforcement patch at the posterior sclera at an area external to the central retina-the macula.

Thus, the present invention provides a synthetic ophthalmic device comprising a porous polymeric structure; wherein said structure is in the shape of a truncated hemisphere having a first top circular opening with a first radius and a hemisphere bottom opening with a second radius and a hemisphere height defined between the centers of said first and second openings; and wherein said porous polymer has pores of less than 5 microns.

In some embodiments, said synthetic ophthalmic device having a porous polymeric structure of the invention has pores of between 5 to 20 microns.

In some further embodiments, a synthetic ophthalmic device of the invention is a biocompatible patch. In some embodiments, a synthetic ophthalmic device of the invention is non-degradable biocompatible patch (i.e. said device does not substantially degrade when being used in a body of a subject). In another embodiments, a synthetic ophthalmic device of the invention is biodegradable biocompatible patch (i.e. said device degrades, breaks or disintegrate after a pre-determined period of time when used in a body of a subject).

In some embodiments, a synthetic ophthalmic device of the invention has a thickness of between 50 to 250 microns. In some other embodiments, said thickness is between 250 to 500 microns.

In some embodiments, a synthetic ophthalmic device of the invention further comprises at least one posterior anchoring band. In some embodiments, a synthetic ophthalmic device of the invention further comprises at least one posterior anchoring band and at least one posterior surface connected thereto. Said posterior anchoring band is connected to said synthetic ophthalmic device at any at least one point of the truncated hemisphere structure. In some embodiments, said posterior anchoring band is connected to said synthetic ophthalmic device at any at least two points of the truncated hemisphere structure, each point of connection is located at the same distance from the centers of said first and second openings. In some embodiments, said posterior anchoring band is made from the same porous polymer of said synthetic ophthalmic device of the invention. In other embodiments, said posterior anchoring band is made from a different porous polymer of said synthetic ophthalmic device of the invention. In other embodiments, said posterior anchoring band is made from a different polymer of said synthetic ophthalmic device of the invention. In other embodiments, said posterior anchoring band is made from a different material of said synthetic ophthalmic device of the invention.

In other embodiments, a synthetic ophthalmic device of the invention further comprises at least one opening on a slant surface area of said frustum.

In some embodiments, said porous polymeric structure comprises at least one polymer.

In other embodiments, said porous polymeric structure comprises nanofibers.

In further embodiments, said porous polymeric structure comprises at least one porous electrospun polymer.

In other embodiments, said porous polymeric structure comprises at least one polymer selected from poly(DTE carbonate) polycaprolactone (PCL), polylactic acid (PLA), poly-L-lactic acid (PLLA), Poly(DL-lactide-co-caprolactone, Poly(ethylene-co-vinyl acetate) vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyurethane, aromatic polyurethane, polycarbonate, polyvinyl chloride (PVC), hyaluronic acid (HA), chitosan, alginate, polyhydroxybuyrate and its copolymers, Nylon 11, Cellulose acetate, hydroxyappetite, poly(3-hydroxybutyric acid-co-3-hydroxyvaleric acid), poly(DL-lactide), polycaprolactone, and poly(L-lactide) or any combination thereof.

In some embodiments, a synthetic ophthalmic device of the invention further comprises at least one active agent. In some embodiments, said at least one active agent is selected from a protein, type I collagen, fibronectin, or TGF-beta 2, heparin, growth factors, antibodies, antimetabolites, chemotherapeutic agents, anti-inflammatory agent, anti-biotic agent, and any combinations thereof.

In some embodiments, said first radius is at least 5 mm. In other embodiments, first radius is in the range of between about 5 to 15 mm. In other embodiments, first radius is about 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0 mm.

In further embodiments, said second radius is at least 10 mm. In other embodiments, said second radius is in the range of between about 10 to 15 mm. In other embodiments, second radius is about 10.0, 10.5, 11.0, 11.5, 12.0, 12.5, 13.0, 13.5, 14.0, 14.5, 15.0 mm.

In some embodiments, said height between first and second radius is at least 1 mm. In further embodiments, said height between first and second radius is in the range of between about 1 to 10 mm.

In a further aspect the invention provides a synthetic ophthalmic device as disclosed herein above and below, for use in the treatment of myopia.

The invention also provides a synthetic ophthalmic device as disclosed herein above and below, for use in slowing the progression of myopia.

When referring to “myopia” it should be understood to encompass the condition defined by nearsightedness being a vision condition in which a subject can clearly see only objects near to subject's eye, but objects farther away are seen as blurry. It occurs when the shape of a subject's eye causes light rays to bend (refract) incorrectly, focusing images in front of subject's retina instead of on the retina.

The term “myopia” also includes the condition known as “high myopia” which is a serious form of the condition, where the eyeball of a subject grows more than it is supposed to and becomes very long front to back. Besides making it hard to see things at a distance, it can also raise the chance of having other conditions like a detached retina, cataracts, and glaucoma.

Furthermore, the term also includes the condition known as “degenerative myopia” (also known as “pathological or malignant myopia”) which is a rare type of hereditary myopia. The eyeball of a subject suffering from degenerative myopia gets longer very quickly and causes severe myopia, usually by the teenage or early adult years. This type of myopia can get worse far into adulthood. Besides making it hard to see things at a distance, a subject may have a higher chance of having a detached retina, abnormal blood vessel growth in the eye (choroid neovascularization), and glaucoma.

The invention further provides a synthetic ophthalmic device as discloses herein above and below, for use in the treatment of at least one disease, condition or symptom selected from Terrien's marginal degeneration, brittle cornea syndrome, Ehlers-Danlos syndrome, osteogenesis imperfecta syndrome, pseudoxanthoma elasticum, congenital cornea plana, scleromalacia perforans, myopia, rheumatoid arthritis (including juvenile RA and adult RA), Marfan syndrome and any combinations thereof.

The invention further provides a method of treating a subject suffering from at least one disease, condition or symptom selected from Terrien's marginal degeneration, brittle cornea syndrome, Ehlers-Danlos syndrome, osteogenesis imperfecta syndrome, pseudoxanthoma elasticum, congenital cornea plana, scleromalacia perforans, myopia, Rheumatoid arthritis, Marfan syndrome and any combinations thereof; said method comprising implanting in the eye of a subject a synthetic ophthalmic device as discloses herein above and below.

In some embodiments, said synthetic ophthalmic device of the invention is used for the treatment of Terrien's marginal degeneration. In some embodiments, said synthetic ophthalmic device of the invention is used for the treatment of brittle cornea syndrome. In some embodiments, said synthetic ophthalmic device of the invention is used for the treatment of Ehlers-Danlos syndrome. In some embodiments, said synthetic ophthalmic device of the invention is used for the treatment of osteogenesis imperfecta syndrome. In some embodiments, said synthetic ophthalmic device of the invention is used for the treatment of pseudoxanthoma elasticum. In some embodiments, said synthetic ophthalmic device of the invention is used for the treatment of congenital cornea plana. In some embodiments, said synthetic ophthalmic device of the invention is used for the treatment of scleromalacia perforans. In some embodiments, said synthetic ophthalmic device of the invention is used for the treatment of rheumatoid arthritis (including juvenile RA and adult RA). In some embodiments, said synthetic ophthalmic device of the invention is used for the treatment of Marfan syndrome.

The term “Terfien's marginal degeneration” refers to a chronic progressive thinning of the peripheral cornea associated with vascularization and lipid deposition. As the disease progresses, marked astigmatism and severe peripheral corneal thinning often develop. Because of the slowly progressive and painless property, the degeneration often has reached an advanced stage when patients visit the hospital, at which the risk of spontaneous or traumatic corneal rupture becomes high. Perforation occurs spontaneously or after minor trauma in 15% of the reported cases. Some severe cases even suffer corneal hydrops or epithelial cysts. Neither spectacles nor contact lenses could correct extreme astigmatism. Reconstructive keratoplasty is required for the severe Terrien's marginal degeneration.

The term “brittle cornea syndrome” (BCS) is a genetic disease involving the connective tissue in the eyes, ears, joints, and skin. The symptoms of BCS typically involve thinning of the protective outer layer of the eye (cornea), which may lead to tearing or rupture after minor damage to the cornea.

The term “Ehlers-Danlos syndrome” is a group of disorders that affect connective tissues supporting the skin, bones, blood vessels, and many other organs and tissues. Defects in connective tissues cause the signs and symptoms of these conditions, which range from mildly loose joints to life-threatening complications. Subjects suffering from this syndrome also display symptoms wherein the eyewall progressively thin to a degree where surgical reinforcement is required.

The term “osteogenesis imperfecta syndrome” (OI) is a group of genetic disorders that mainly affect the bones. Subjects suffering from this syndrome also display weakening eyeball structure due to damaged collagen.

The term “pseudoxanthoma elasticum” (PXE) is a progressive disorder that is characterized by the accumulation of deposits of calcium and other minerals (mineralization) in elastic fibers. Elastic fibers are a component of connective tissue, which provides strength and flexibility to structures throughout the body. Subjects suffering from this disorder display ocular findings ranging from retinal damage, angioid streaks and blue sclera. The eyewall will benefit from fortification as most of the findings are associated with thinning of the sclera.

The term “congenital cornea plana” (CNA2) is an extremely rare congenital hereditary deformity of the eye surface, leading to severe decrease in corneal curvature. There is evidence that cornea plana 2 is caused by mutations in KERA gene encoding keratocan.

The term “scleromalacia perforans” refers to is a rare ocular manifestation of rheumatoid arthritis which can potentially lead to blindness and is a late consequence in the course of the disease.

The term “Marfan syndrome” refers to a genetic disorder that affects the connective tissue. Those with the condition tend to be tall and thin, with long arms, legs, fingers and toes. Subjects suffering from this condition also typically have overly-flexible joints and scoliosis. Most people with Marfan syndrome suffer from nearsightedness, or myopia, and abnormal curvature of the eye, or astigmatism. These can be notably high since the connective tissue defect can affect the cornea, lens, and growth of the eye.

Rheumatoid arthritis is an autoimmune condition in which the immune system mistakenly targets the joints. Rheumatoid arthritis causes inflammation and is a systemic disease meaning it can affect the entire body. As it relates to the eye, rheumatoid arthritis may lead to conditions such as dry eye, scleritis, or uveitis. Dry eye is a common condition in which the eyes do not produce adequate tears, leading to redness and irritation. Scleritis affects the sclera, or white portion of the eye, causing pain and inflammation. Uveitis is an inflammatory condition that affects the uvea, or inner portion of the eye. It was found that there is a higher incidence of myopia among patients with inflammatory diseases such as type 1 diabetes mellitus (7.9%), uveitis (3.7%), or systemic lupus erythematosus (3.5%) compared to those without inflammatory diseases.

The term “treatment” as used herein refers to use of a synthetic ophthalmic device of the invention which is effective to ameliorate listed conditions, diseases or syndromes, to prevent the manifestation of such listed conditions, diseases or syndromes before they occur, to slow down the progression of the listed conditions, diseases or syndromes, slow down the deterioration of listed conditions, diseases or syndromes, slow down the irreversible damage caused in the progressive listed conditions, diseases or syndromes, to delay the onset of said progressive stage of listed conditions, diseases or syndromes, to lessen the severity or cure the listed conditions, diseases or syndromes, or to prevent the listed conditions, diseases or syndromes form occurring or a combination of any of the above.

As used herein, the term “subject” or “patient” or “individual”, such as the subject in need of treatment listed herein, may be a human of any age group including for example: a new born (a subject 0-6 months old including a premature baby), an infant (a subject 0-1 years old), a toddler (a subject 1-10 years old), an adolescent (a subject 10-18 years old), an adult (a subject 18-65 years old), an elderly (a subject above 65 years old).

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows an embodiment of a device of the invention.

It will be appreciated that for simplicity and clarity of illustration, elements shown in the FIGURES have not necessarily been drawn to scale. For example, the dimensions of some of the elements may be exaggerated relative to other elements for clarity. Further, where considered appropriate, reference numerals may be repeated among the FIGURES to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

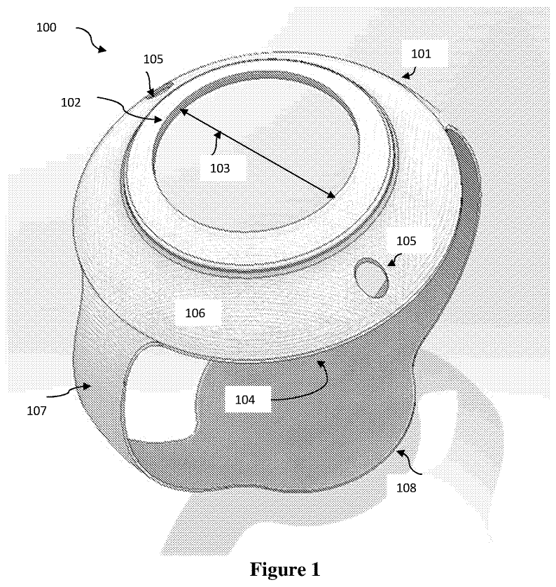

FIG. 1 shows an embodiment of a synthetic ophthalmic device of the invention (100). Said device comprises a porous polymeric structure in the shape of a truncated hemisphere (101) having a first top circular opening (102) with a first radius (103) and a hemisphere bottom opening (104) with a second radius (not shown) and a hemisphere height defined between the centers of said first and second openings (not shown); and wherein said porous polymer has pores of less than 5 microns. Said device further comprises two openings (105) on a slant surface area (106) of said truncated hemisphere. Additionally, the device shown in FIG. 1 also comprises a posterior anchoring band (107) having a posterior anchoring surface (108).

One embodiment can be a truncated hemisphere covering the anterior sclera overlying the insertion of the ciliary body. Rabbit trials will be carried out and compared to a non-operated eye. We expect to see a lesser elongation of the implanted eye thus proving the mechanism of action. Rabbit trials carried out to date prove seamless integration Basing biocompatibility and safety.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A synthetic ophthalmic device comprising a porous polymeric structure; wherein said structure is in the shape of a truncated hemisphere having a first top circular opening with a first radius and a hemisphere bottom opening with a second radius and a hemisphere height defined between the centers of said first and second openings; and wherein said porous polymer has pores of less than 5 microns.

2. A synthetic ophthalmic according to claim 1, device having a porous polymeric structure with pores of between 100 nanometers to 5 micrometers.

3. A synthetic ophthalmic device according to claim 1, having a porous polymeric structure with pores of between 5 to 20 microns.

4. (canceled)

5. A synthetic ophthalmic device according to claim 1, being a non-degradable biocompatible patch.

6. A synthetic ophthalmic device according to claim 1, being a biodegradable biocompatible patch.

7. A synthetic ophthalmic device according to claim 1, having a thickness of between 50 to 250 microns.

8. A synthetic ophthalmic device according to claim 1, having a thickness of between 250 to 500 microns.

9. A synthetic ophthalmic device according to claim 1, further comprising a posterior anchoring band.

10. A synthetic ophthalmic device according to claim 1, further comprises at least one opening on a slant surface area of said truncated hemisphere.

11. (canceled)

12. A synthetic ophthalmic device according to claim 1, wherein said porous polymeric structure comprises nanofibers.

13. A synthetic ophthalmic device according to claim 1, wherein said porous polymeric structure comprises at least one porous electrospun polymer.

14. A synthetic ophthalmic device according to claim 1, wherein said porous polymeric structure comprises at least one polymer selected from poly(D carbonate) polycaprolactone (PCL), polylactic acid (PLA), poly-L-lactic acid (PLLA), Poly(DL-lactide-co-caprolactone, Poly (ethylene-co-vinyl acetate) vinyl acetate, Poly(methyl methacrylate), Poly(propylene carbonate), Poly(vinylidene fluoride), Polyacrylonitrile, Polycaprolactone, Polycarbomethylsilane, Polylactic acid, Polystyrene, Polyvinylpyrrolidone, poly vinyl alcohol (PVA), polyethylene oxide (PEO), polyurethane, aromatic polyurethane, polycarbonate, polyvinyl chloride (PVC), hyaluronic acid (HA), chitosan, alginate, polyhydroxybuyrate and its copolymers, Nylon 11, Cellulose acetate, hydroxyappetite, poly(3-hydroxybutyric acid-co-3-hydroxyvaleric acid), poly(DL-lactide), polycaprolactone, and poly(L-lactide) or any combination thereof.

15. A synthetic ophthalmic device according to claim 1, further comprising at least one active agent.

16. (canceled)

17. A synthetic ophthalmic device according to claim 1, wherein said first radius is at least 5 mm.

18. A synthetic ophthalmic device according to claim 1, wherein said first radius is in the range of between about 5 to 15 mm.

19. A synthetic ophthalmic device according to claim 1, wherein said second radius is in the range of between about 7 to 13 mm.

20. A synthetic ophthalmic device according to claim 1, wherein said height between first and second radius is at least 1 mm.

21. A synthetic ophthalmic device according to claim 1, wherein said height between first and second radius is in the range of between about 1 to 10 mm.

22. A method of treatment and/or slowing the progression of myopia and any disease, disorder, condition or symptom associated therewith, said method comprising the step of transplanting into the eye of a subject in need thereof a synthetic ophthalmic device comprising a porous polymeric structure; wherein said structure is in the shape of a truncated hemisphere having a first top circular opening with a first radius and a hemisphere bottom opening with a second radius and a hemisphere height defined between the centers of said first and second openings; and wherein said porous polymer has pores of less than 5 microns.

23. (canceled)

24. A method according to claim 22, wherein said disease, disorder, condition or symptom associate with myopia is selected from Terrien's marginal degeneration, brittle cornea syndrome, Ehlers-Danlos syndrome, osteogenesis imperfecta syndrome, pseudoxanthoma elasticum, congenital cornea plana, scleromalacia perforans, myopia, Rheumatoid arthritis, Marfan syndrome and any combinations thereof; said method comprising the step of transplanting into the eye of a subject in need thereof a synthetic ophthalmic device comprising a porous polymeric structure; wherein said structure is in the shape of a truncated hemisphere having a first top circular opening with a first radius and a hemisphere bottom opening with a second radius and a hemisphere height defined between the centers of said first and second openings; and wherein said porous polymer has pores of less than 5 microns.
